(19) United States
(12) Patent Application Publication
(10) Pub. No.: US 2015/0065681 A1
(43) Pub. Date: Mar. 5, 2015

(54) Method of Preparing Prepolymer Particle

(71) Applicant: Samsung SDI Co., Ltd, Yongin-si (KR)

(72) Inventors: Jin Kyu KIM, Uiwang-si (KR); So Young KWON, Uiwang-si (KR); Sang Kyun IM, Uiwang-si (KR); Ki Yon LEE, Uiwang-si (KR); Suk Min JUN, Uiwang-si (KR); Gook Geon BAE, Uiwang-si (KR)

(21) Appl. No.: 14/465,860

(22) Filed: Aug. 22, 2014

(30) Foreign Application Priority Data
Sep. 2, 2013 (KR) 10-2013-0105143

(51) Int. Cl. C08G 69/28; C07C 231/02; C07C 69/96; C07C 67/00; C07C 69/78; C07C 68/00; C08G 69/26; C07C 233/76

(52) U.S. Cl. CPC C08G 69/28; C08G 69/265; C07C 231/02; C07C 233/76; C07C 67/00; C07C 69/78; C07C 68/00; C07C 69/96
USPC 528/339; 564/139; 564/153; 560/85; 558/274; 422/131

(57) ABSTRACT

Disclosed herein is a method of preparing prepolymer particles. The method includes injecting a prepolymer in a solution state by bottom-up type injection to form prepolymer particles by removing a solvent from the prepolymer in a solution state. The method can prepare prepolymer particles, which can be used in solid-state polymerization, using a bottom-up type flash process.

### CROSS-REFERENCE TO RELATED APPLICATION

This application claims priority under 35 USC Section 119 to and the benefit of Korean Patent Application No. 10-2013-0105143, filed Sep. 2, 2013, the entire disclosure of which is incorporated herein by reference.

### FIELD OF THE INVENTION

The present invention relates to a method of preparing prepolymer particles. More particularly, the present invention relates to a method of preparing prepolymer particles, which can be used in solid-state polymerization, using a bottom-up type flash method.

### BACKGROUND

In an aerosol process or a flash process as a widely known polymerization method, a low molecular weight polymer is polymerized in a solution state and then subjected to particlization (crystallization) through injection from high pressure to normal pressure. More particularly, the flash process includes subjecting a polymer (prepolymer in a liquid state), which has been polymerized to a certain degree under high temperature and high pressure conditions, to normal pressure injection through an orifice or the like to evaporate a solvent used in polymer polymerization, and collecting a particlized (granulized) polymer, from which the solvent has been evaporated. Such a flash process can be modified depending upon properties of each polymer. Specifically, in a highly thermal resistant nylon polymerization process, the flash process may be used as a process for preparing prepolymer particles (granules) to be used in solid-state polymerization. Since the prepolymer particles have a relatively low molecular weight, the preparation of the prepolymer particles requires more careful process conditions.

U.S. Pat. No. 4,603,193 discloses a flash process in which a prepolymer in a solution state is injected by top-down type injection and dried through a wall surface of a flash reactor at high temperature to improve drying efficiency. Such a flash process has an advantage in that a solvent used can be efficiently removed from the prepolymer. However, there is a concern in that a condensation polymer such as a nylon resin and the like can suffer from discoloring upon contact with oxygen at high temperature. Further, the condensation polymer is highly likely to melt, carbonize or degrade. In this regard, the prepared prepolymer requires post treatment to be used in solid-state polymerization. Therefore, there is a need for a method of preparing prepolymer particles capable of suppressing or preventing the occurrence of discoloring, melting, carbonization, and degradation of the prepolymer particles (granules) without additional processing.

### SUMMARY

The present invention can provide a method of preparing prepolymer particles capable of being used in solid state polymerization, which can suppress or prevent the occurrence of discoloring, melting, carbonization and degradation of prepolymer particles when preparing the prepolymer particles from a prepolymer in a solution state, prepolymer particles prepared using the method, and a flash reactor for preparing prepolymer particles.

In exemplary embodiments, the method of preparing prepolymer particles includes injecting a prepolymer in a solution state by bottom-up type injection to form prepolymer particles by removing a solvent from the prepolymer in a solution state.

In one embodiment, the prepolymer in a solution state is injected by bottom-up type injection under conditions of about 200° C. to about 300° C. and about 10 bar to about 40 bar through a bottom nozzle provided to a flash reactor under conditions of about 15° C. to about 30° C. and about 0 bar to about 3 bar.

In one embodiment, the prepolymer in a solution state may have an intrinsic viscosity from about 0.05 dL/g to about 0.2 dL/g.

In one embodiment, the prepolymer in a solution state may be injected at an injection rate from about 10 m/sec to about 70 m/sec.

In one embodiment, the flash reactor may have a height from about 3 m to about 30 m and a volume from about 1 m³ to about 20 m³.

In one embodiment, the prepolymer particle may have an intrinsic viscosity from about 0.05 dL/g to about 0.2 dL/g and a weight average molecular weight from about 500 g/mol to about 3,000 g/mol.

Exemplary embodiments of the present invention also relate to prepolymer particles. The prepolymer particles are prepared by the method of preparing prepolymer particles.

The present invention further relates to a flash reactor. The flash reactor may be provided at a bottom thereof a nozzle capable of injecting a prepolymer in a solution state in a way of bottom-up type injection.

In one embodiment, the flash reactor may have a height from about 3 m to about 30 m and a volume from about 1 m³ to about 20 m³.

In one embodiment, the prepolymer in a solution state may be injected at an injection rate from about 10 m/sec to about 70 m/sec.

In one embodiment, the flash reactor may have an inner temperature from about 15° C. to about 30° C. and a pressure from about 0 bar to about 3 bar.

### BRIEF DESCRIPTION OF DRAWINGS

FIG. 1 is a schematic view of a flash reactor used in a method of preparing prepolymer particles in accordance with one embodiment of the present invention.

### DETAILED DESCRIPTION

Exemplary embodiments now will be described more fully hereinafter in the following detailed description with reference to the accompanying drawing, in which some, but not all embodiments of the invention are described. Indeed, this invention may be embodied in many different forms and should not be construed as limited to the embodiments set forth herein; rather, these embodiments are provided so that this disclosure will satisfy applicable legal requirements. In the drawing, the dimensions of may be exaggerated for clarity of illustration. Like reference numerals refer to like elements throughout.

According to one embodiment of the present invention, a method of preparing prepolymer particles may include injecting a prepolymer in a solution state by bottom-up type injection to form prepolymer particles by removing a solvent from the prepolymer in a solution state.

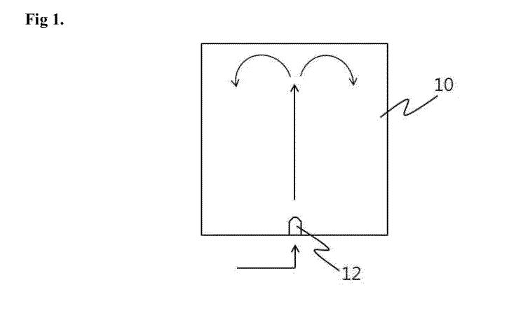

FIG. 1 is a schematic view of a flash reactor used in the method of preparing prepolymer particles in accordance with one embodiment of the invention. As shown in FIG. 1, the prepolymer in a solution state may be injected through a bottom nozzle 12 placed at the bottom of a flash reactor 10 by bottom-up type injection.

In one embodiment, the method of preparing prepolymer particles may include injecting a prepolymer in a solution state under conditions of about 200° C. to about 300° C. and about 10 bar to about 40 bar through the bottom nozzle 12 provided to the flash reactor 10 under conditions of about 15° C. to about 30° C. and about 0 bar to about 3 bar by bottom-up type injection.

According to the present invention, the prepolymer may be a prepolymer of resins prepared by condensation polymerization, for example, aliphatic polyamide polymers such as Nylon 66, Nylon 6, and the like, aromatic polyamide polymers (high heat resistant nylon resin) such as Nylon 6T, Nylon 9T, Nylon 10T, Nylon 12T, and the like, polyester resins such as polyethylene terephthalate (PET) and the like, polycarbonate (PC) resins, and the like, and combinations thereof, without being limited thereto. In exemplary embodiments, the prepolymer is a prepolymer of the high heat resistant nylon resin.

The prepolymer in a solution state used in the present invention may be obtained by typical condensation polymerization. Although preparation conditions can be changed depending upon the kind of polymer (resin) to be prepared, the prepolymer in a solution state may be prepared by reacting monomers of a desired prepolymer at a temperature of about 200° C. to about 300° C., for example, about 200° C. to about 250° C. and at a pressure of about 10 bar to about 40 bar, for example, about 15 bar to about 35 bar, about 15 bar to about 30 bar. For example, in the case of preparing a polyamide polymer, the prepolymer in a solution state may be a solution containing water, a prepolymer obtained by condensation polymerization of monomers including a dicarboxylic acid such as terephthalic acid and a diamine such as hexamethylene diamine in a molar ratio of about 0.9:about 1.0 to about 1.1:about 1.0 (dicarboxylic acid:diamine), and the like.

Although the prepolymer in a solution state can have a different intrinsic viscosity depending upon a polymer to be prepared, in one embodiment, the prepolymer in a solution state can have an intrinsic viscosity from about 0.05 dL/g to about 0.2 dL/g, for example, from about 0.1 dL/g to about 0.2 dL/g. The intrinsic viscosity is measured at 25° C. using an Ubbelohde Viscometer after dissolving the prepolymer in concentrated sulfuric acid (about 98%).

Further, although a solvent included in the prepolymer in a solution state can be different depending upon the polymer to be prepared, examples of the solvent can include without limitation water, alcohols such as methanol, ethanol, isopropyl alcohol, and the like, aromatic solvents, typical organic solvents such as ketones, glycol ethers, acetates, and the like, and mixtures thereof.

Referring to FIG. 1, the flash reactor 10 may include a bottom nozzle 12, through which the prepolymer in a solution state is injected by bottom-up type injection. In a conventional flash reactor, the prepolymer in a solution state is injected by top-down type injection and the solvent is removed from the prepolymer by heating. In contrast, in the method according to the present invention, a flash reactor 10 can be used at a temperature of about 15° C. to about 30° C., for example, about 20° C. to about 25° C., to inject the prepolymer in a solution state by bottom-up type injection, thereby allowing the prepolymer particles to be cooled for a sufficient period of time. When the temperature of the flash reactor 10 is greater than about 30° C., the prepolymer particles can suffer from discoloring, melting, carbonization or degradation. When the temperature of the flash reactor 10 is less than about 15° C., there is a concern of insufficient removal of the solvent from the prepolymer.

Further, the flash reactor 10 can be maintained at a pressure of about 0 bar to about 3 bar, for example, about 0 bar to about 2 bar, and as another example, about 1 bar to about 1.5 bar (reduced pressure or normal pressure), such that the solvent can be removed (flashed) from the injected prepolymer in a solution state under high temperature and high pressure conditions to form prepolymer particles (granules). When the inner pressure of the flash reactor 10 is greater than about 3 bar, there is a concern of insufficient removal of the solvent from the prepolymer in a solution state. When the inner pressure of the flash reactor 10 is less than about 0 bar, there is a concern in that the prepolymer can suffer from discoloring due to inclusion of oxygen and the like.

In one embodiment, the prepolymer in a solution state may be injected at an injection rate from about 10 m/sec to about 70 m/sec, for example, from about 15 m/sec to about 60 m/sec, and as another example, from about 20 m/sec to about 50 m/sec. Within this range, solvent removal and cooling time can be sufficiently secured.

In one embodiment, the flash reactor 10 may have various shapes such as a cylindrical shape, a square shape, a rectangular shape, and the like, depending upon the kinds of prepolymer particles to be prepared. For example, the flash reactor 10 may have a height from about 3 m to about 30 m, for example, from about 3 m to about 10 m, and a volume from about 1 m³ to about 20 m³, for example, from about 1 m³ to about 10 m³. Within this range, the solvent removal and cooling time can be sufficiently secured.

The prepolymer particle of the present invention may have a wet cake shape, in which a low-order prepolymer prepared by the flash process has various shapes, such as particles, granules, powder, and/or chips, and various sizes (for example, about 1 μm to about 50 mm), and can include about 1 wt % to about 15 wt % of moisture and/or organic solvents, without being limited thereto. For example, the prepolymer particles may have an intrinsic viscosity (IV) from about 0.05 dL/g to about 0.2 dL/g, as measured using an Ubbelohde Viscometer at 25° C. after dissolving the particles in a concentrated sulfuric acid solution (about 98%). For example, the prepolymer may have an intrinsic viscosity from about 0.1 dL/g to about 0.2 dL/g.

Further, the prepolymer particle may have a weight average molecular weight from about 500 g/mol to about 3,000 g/mol, as determined by Gel Permeation Chromatography (GPC), without being limited thereto. For example, the prepolymer may have a weight average molecular weight from about 1,000 g/mol to about 1,500 g/mol.

The method of preparing prepolymer particles according to the present invention employs a bottom-up type flash process, which can provide sufficient cooling time of the prepolymer particles. For this reason, it is possible to reduce or suppress discoloring, melting, carbonization or degradation of the prepolymer particles due to the temperature for heating the flash reactor and latent heat of the final prepolymer particles.

Accordingly, the method of preparing prepolymer particles according to the present invention can omit post-treatment after preparation of the prepolymer particle, thereby providing economical feasibility. When using the prepolymer particle in solid-state polymerization, a finally obtained polymer can have good yield and color.

Next, the present invention will be described in more detail with reference to the following examples. It should be understood that these examples are provided for illustration only and are not to be construed in any way as limiting the present invention.

Descriptions of details apparent to those skilled in the art will be omitted herein.

### EXAMPLES

### Examples 1 to 3

To a 1 liter autoclave, terephthalic acid (TPA), adipic acid (AA) and isophthalic acid (IPA) as dicarboxylic acid components and 1,6-hexamethylene diamine (HDMA) as a diamine component are added in amounts as listed in Table 1, followed by adding 1.5 parts by mole of acetic acid as an end capping agent based on 100 parts by mole of the dicarboxylic acid components and the diamine component, 0.1 parts by weight of sodium hypophosphinate as a catalyst and 38 parts by weight of water based on 100 parts by weight of the dicarboxylic acid components and the diamine component, and then purged with nitrogen. Next, the resultant material is stirred at 210° C. for 60 minutes, followed by warming up to 230° C. for 1 hour, and reacted for 1 hour while maintaining a pressure of 20 bar to 30 bar to prepare a prepolymer in a solution state. Subsequently, the prepolymer in a solution state is injected at an injection rate of 40 m/sec through a nozzle at the bottom of a flash reactor (inner temperature: 25° C., inner pressure: 0 bar to 2 bar, cylindrical shape, height: 5 m, volume: 4 m³, see FIG. 1) by bottom-up type injection, thereby removing the solvent (water) from the prepolymer to prepare prepolymer particles.

### Comparative Examples 1 to 3

Prepolymer particles are prepared in the same manner as in Example 1 except that a prepolymer in a solution state is injected at an injection rate of 40 m/sec through a nozzle at the top of a flash reactor (inner temperature: 25° C., inner pressure: 0 bar to 2 bar, cylinder shape, height: 5 m, volume: 4 m³) by top-down type injection.

TABLE1

| | | Example | | | Comparative Example | | |
|---|---|---|---|---|---|---|---|
| Monomer | | 1 | 2 | 3 | 1 | 2 | 3 |
| Diacid | TPA (% by mole) | 55 | 65 | 65 | 55 | 65 | 65 |
| | AA (% by mole) | 45 | 10 | 35 | 45 | 10 | 35 |
| | IPA (% by mole) | — | 25 | — | — | 25 | — |
| Diamine | HMDA (% by mole) | 100 | 100 | 100 | 100 | 100 | 100 |
| Molar ratio | [Diamine]/ | 1.013 | | | | | |
| | [Diacid] | | | | | | |

### Experimental Example

Intrinsic viscosity, bulk density and color of the prepared prepolymer particles are measured and also the prepolymer particles are checked to determine whether there is melting and carbonization of the prepolymer particles. Results are shown in Table 2.

Evaluation of Physical Properties

(1) Intrinsic viscosity (V) (unit: dL/g): After dissolving the prepolymer particles in a sulfuric acid solution (98%), intrinsic viscosity is measured at 25° C. using an Ubbelohde Viscometer.

(2) Bulk density (unit: g/ml): The weight of the prepolymer particles filling a 100 ml mass cylinder is measured.

(3) Average particle size: The average particle size of the prepolymer particles is measured using an optical microscope.

(4) Color: Whiteness (L*) and yellowness (b*) are measured using a Minolta 3600D CIE Lab. Colorimeter.

(5) Melting and carbonization phenomena: Melting and carbonization of the prepolymer particles are observed with the naked eye.

TABLE 2

| | | Example 1 | Example 2 | Example 3 | Com. Ex. 1 | Com. Ex. 2 | Com. Ex. 3 |
|---|---|---|---|---|---|---|---|
| Intrinsic viscosity (dL/g) | | 0.13 | 0.12 | 0.13 | 0.13 | 0.14 | 0.12 |
| Bulk density (g/ml) | | 0.05 | 0.048 | 0.055 | NM | NM | NM |
| Color | Whiteness (L*) | 95 | 94 | 96 | 88 | 86 | 89 |
| | Yellowness (b*) | 6 | 5 | 7 | 12 | 13 | 10 |

| | | | | | | |
|---|---|---|---|---|---|---|
| Average particle size | 48 μm | 66 μm | 55 μm | NM | NM | NM |
| Melting and carbonization | None | None | None | Yes | Yes | Yes |
| phenomena | | | | | | |

Note:
“NM” means “not measurable”

In Table 2, it can be seen that, in the case of preparing prepolymer particles by the method of preparing prepolymer particles through bottom-up type injection according to the present invention, prepolymer particles having a desired intrinsic viscosity could be prepared without deterioration in colors (whiteness and yellowness). In addition, it can be seen from the results of bulk density that the prepolymer is not melted, thereby providing a prepolymer in particle form.

In contrast, it can be seen that, in the case of preparing prepolymer particles by a typical method of preparing prepolymer particles through top-down type injection, although the prepolymer has a desired intrinsic viscosity, the prepolymer suffers from deterioration in color due to carbonization and is melted. Thus, it is difficult to obtain the prepolymer in particle form.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims.

What is claimed is:

1. A method of preparing prepolymer particles, comprising injecting a prepolymer in a solution state by bottom-up type injection to form prepolymer particles by removing a solvent from the prepolymer in a solution state.

2. The method according to claim 1, wherein the prepolymer in a solution state is injected by bottom-up type injection under conditions of about 200° C. to about 300° C. and about 10 bar to about 40 bar through a bottom nozzle provided to a flash reactor under conditions of about 15° C. to about 30° C. and about 0 bar to about 3 bar.

3. The method according to claim 1, wherein the prepolymer in a solution state has an intrinsic viscosity from about 0.05 dL/g to about 0.2 dL/g.

4. The method according to claim 1, wherein the prepolymer in a solution state is injected at an injection rate from about 10 m/sec to about 70 m/sec.

5. The method according to claim 1, wherein the flash reactor has a height from about 3 m to about 30 m and a volume from about 1 m³ to about 20 m³.

6. The method according to claim 1, wherein the prepolymer particle has an intrinsic viscosity from about 0.05 dL/g to about 0.2 dL/g, and a weight average molecular weight from about 500 g/mol to about 3,000 g/mol.

7. A prepolymer particle prepared by the method of preparing prepolymer particles according to claim 1.

8. A flash reactor comprising a bottom nozzle for injecting a prepolymer in a solution state by bottom-up type injection.

9. The flash reactor according to claim 8, wherein the flash reactor has a height from about 3 m to about 30 m and a volume from about 1 m³ to about 20 m³.

10. The flash reactor according to claim 8, wherein the prepolymer in a solution state is injected at an injection rate from about 10 m/sec to about 70 m/sec.

11. The flash reactor according to claim 8, the flash reactor has an inner temperature from about 15° C. to about 30° C. and a pressure from about 0 bar to about 3 bar.
